(19) United States
(12) Patent Application Publication
(10) Pub. No.: US 2013/0041329 A1
(43) Pub. Date: Feb. 14, 2013

(54) NASAL RINSING DEVICE

(71) Applicants: Gerhart P. HUY, Hamilton Square, NJ (US); Matthew Kwang, Old Bridge, NJ (US)

(72) Inventors: Gerhart P. HUY, Hamilton Square, NJ (US); Matthew Kwang, Old Bridge, NJ (US)

(73) Assignee: CHURCH & DWIGHT CO., INC., Princeton, NJ (US)

(21) Appl. No.: 13/205,978

(22) Filed: Aug. 9, 2011

(51) Int. Cl. A61M 3/02

(52) U.S. Cl. 604/257

(57) ABSTRACT

The present invention is directed towards a nasal rinsing device also known as a neti pot. The neti pot comprises a container body for holding liquid, a removable cap, and an extended pouring spout that directs a saline solution into nasal passages. The cap provides an adequate liquid-seal and venting for airflow into the interior of the pot.

### FIELD OF THE INVENTION

This invention is directed towards an apparatus for rinsing nasal passages.

### BACKGROUND

The nose is a very specialized and complicated structure that serves dual functions as the organ for the sense of smell and as an entrance to the respiratory tract. As a part of the respiratory tract, a healthy nose moisturizes and warms incoming air and filters out foreign materials.

Nasal passages and other portions of the respiratory tract are lined with specialized tissue layers. In the nose and sinus areas this tissue is often called the nasal mucosa. Like many tissues, the nasal mucosa is composed of several cell layers and cell types. Mucous cells are located throughout the nasal mucosa and are generally clustered into small glands. These glands secrete a sticky substance called mucus. Mucus is composed of water, shed epithelial (surface) cells, dead leukocytes, mucin, and inorganic salts, among other things, that are all held in suspension. Mucus functions as a trap for airborne particles (e.g., dust, bacteria, and viruses) that enter the nasal passages. Mucus also lubricates the walls of the nose, sinuses, and throat.

In a healthy nose, the mucus is cleared from the nasal passages on a regular basis by a layer of cells in the nasal mucosa called the ciliated columnar epithelium. These cells possess small hair-like projections called cilia that undulate and “sweep” mucus through the nasal passages allowing it to drain to the back of the throat where it can be swallowed or expelled. This line of defense protects the body against the bacteria and viruses that continually enter the nose and mouth.

Connected to the nose are sinuses or air-filled cavities located behind certain facial bones. There are four groups of sinuses, namely, frontal, sphenoidal, ethmoidal, and maxillary. The sinuses are also lined with mucus secreting tissue. The sinuses are normally kept clear when mucus drains through them into the nasal passages. If they are obstructed for any reason, such as from the congestion present during a cold, normal drainage may not occur and infection of the sinuses may result.

Virtually all persons are occasionally stricken with acute upper respiratory infections (common colds), acute or chronic allergy flare-ups of the nose, and/or acute or chronic non-allergic rhinosinusitis. Persons afflicted by such conditions usually incur significant discomfort and inconvenience.

All of these disorders are characterized by intense inflammation of the nasal mucosa. A number of symptoms contribute to the discomfort and inconvenience associated with the common cold or sinusitis. Symptoms often include one or more of the following: nasal congestion, post-nasal drip, decreased sense of smell, ear fullness, headache, sore throat, malaise, muscle and joint aches, fatigue, cough, chest congestion, fever, chills and gastrointestinal maladies. Considerable research has been conducted over the years aimed at reducing the incidence and duration of symptoms associated with allergies and common colds.

Perhaps the most common symptom experienced by cold and allergy sufferers is “congestion.” The term “congestion” is commonly used by the layperson and in the patent literature as a catchall term to describe a complex series of events that hinder a person's ability to breathe through the nose when they have a cold, sinusitis or an allergic reaction.

For example, “congestion” can include a physiological response to an irritant that involves increased blood flow to tissues lining the nose. This increased blood flow causes these tissues to swell and physically block the nasal passages. Alternatively, when a nose is irritated or injured, fluid can seep into the free spaces in the tissues of the nose or build up in individual cells thereby causing additional “congestion”. “Congestion” can also include the overproduction of mucus or the buildup of mucus due to increased mucus viscosity or depressed cilia activity or both. “Congestion” may also occur when a person lies on his or her side causing receptor cells in the nose to close off one nasal airway. Changes in temperature and/or humidity can alter the tissue of the nose and cause a “congested” feeling.

There are various methods to treat the symptoms of or to cure sinus disease, including surgery. Saline nasal irrigations have been used for many years and have been mentioned in medical textbooks going back hundreds of years. A wide variety of techniques have been described, including swimming in salt water, which often results in some degree of inadvertent nasal salt water irrigation. Alternatively, an effective nasal rinse can significantly reduce or permanently cure the symptoms of nasal allergies and sinus disease.

Nasal rinsing or lavaging is a treatment for rhinitis and sinusitis. Nasal rinsing comprises the dispensing of a saline solution into nasal passages, in order to cleanse and wash away mucus and allergy that create particles and irritants. Nasal rinsing allows the sinuses to drain normally and reduces the inflammation of the mucus membrane.

While saline solutions can easily be prepared at home, consumers often purchase nasal rinsing devices. Conventional nasal rinsing devices can be crude, expensive, and/or lack a proper opening that fits into the user's nostril to dispense the liquid.

Nasal rinsing devices available commercially can be used to both prepare and dispense the saline solution. Typically, a dry mixture of salt and water are added into the device, which is then shaken with its top opening closed with a lid or a cap. The mixture constitutes a ready-to-use saline, which is then dispensed from a spout to rinse nasal passages. The device requires an additional opening above the saline level to allow a sufficient flow of air, so that the air pressures the saline solution to exit the device.

Currently there are several nasal rinsing devices on the market, one of which is known as “NasaFlo®” by NeilMed®. NasaFlo® includes a teapot-like vessel, a spout and a lid. The lid contains a hole to let in air, which then pressures the saline solution to exit the vessel through the spout. Although such design immediately solves the problem of insufficient air to pressure liquid to exit the spout, the location of the hole can create a spillage when the vessel is shaken by hand.

Another example is a neti pot by Waterpik®. The neti pot is a teapot-like vessel, including a handle, a spout and a lid. Additionally, it has a One Touch Flow Control™ vent located atop the handle. The user alternatively covers or uncovers the vent to disallow or allow air to enter the vessel, so to control the flow of liquid through the spout. Although such design gives the consumer an option to cover the vent, it is not possible to maintain a full grip of the handle because as the user pours the solution from the spout, the user has to constantly move his or her thumb to control the liquid flow. Furthermore, the user needs both hands to shake the vessel (to cover the hole with the thumb) and to cover the spout at the same time. Such requirement limits the applied force and user's arm movements, as compared with shaking with one hand.

For the foregoing reasons, there is a need for a nasal rinsing device that enables optimal shaking, as well as provides proper venting and a sufficient grip. The device also needs to maintain an adequate liquid seal such that a minimal or no spillage occurs when the device is shaken by hand, or when the device is positioned in a downward angle. The nasal rinsing device may contain a spout for delivering the solution into nostril of varying sizes.

### SUMMARY OF THE INVENTION

The present invention is directed towards a nasal rinsing device also known as a neti pot. The neti pot comprises a container body for holding liquid, a handle attached to the body, a removable cap, and an extended pouring spout that directs a saline solution into nasal passages. The neti pot further contains an opening that is fitted with a removable cap to provide an adequate liquid seal. The cap and/or neck of the body provides venting for air to enter the pot and provide ease of liquid entry into the nasal cavity.

### BRIEF DESCRIPTION OF THE DRAWINGS

FIG. 1 is a side view of a neti pot.

FIG. 2 is a bottom perspective view of the cap.

FIG. 3 is a partial side elevation view of the cap incorporated onto the neti pot.

FIG. 4 is a cross sectional view of the cap and neck of FIG. 3, and which shows air flow into the opening of the neti pot.

### DETAILED DESCRIPTION OF THE INVENTION

The present invention is directed towards a nasal rinsing device also known as a neti pot. The neti pot comprises a container body for holding liquid, an opening, a platform located circumferentially around the opening, an extended pouring spout that directs a saline solution into nasal passages, and a removable cap. The neti pot body has a threaded neck portion that is perpendicular to the platform, and circumferentially surrounds the opening. The neck includes a spiral thread. Along the spiral thread, optionally, at least one indention is provided. The cap closes the opening of the neti pot. The cap has an outer rim and an inner circular rim that is perpendicular to the interior of the cap. A plurality of venting protrusions extend downward from the edge of either or both of the outer rim and the inner circular rim. The venting protrusions contact the body platform when the cap is threaded onto the neck, preventing a tight seal between the bottom edge of the cap and body platform. The protrusions and, optionally, at least one indention along the neck thread vent the neti pot to allow airflow into the interior of the pot, and still provide an adequate liquid seal to prevent spillage.

The neti pot comprises a container body for holding liquid, an opening, a platform located circumferentially around the opening, and an extended pouring spout that directs a saline solution into a nasal cavity. The neti pot also has a threaded neck that circumferentially surrounds the opening and is perpendicular to the platform. The threaded neck includes a thread which optionally contains at least one indention, such that the indention disrupts the seal against a matching thread on a removable cap. The indention is about 2 to 0.5 mm in length, preferably 1 mm in length. The indention has a width which coincides with the width of the thread. The number of indentions is optimized so when the cap closes the opening, at least one indention provides a channel to allow air to enter the container body. The number of indentions is also optimized so that when the cap closes the opening, the cap adequately seals the opening to prevent spillage.

The body of neti pot can be inflexible and the saline solution dispensed through the spout by gravity. Alternatively, the neti pot can be flexible, and its sidewalls can be easily compressed by hand to force the saline solution through the spout. The neti pot can be made of materials selected from low-density polypropylene, high-density polypropylene, high-density polyethylene, wood, metal, ceramic, or any material that imparts sturdiness for the neti pot to be held and shaken by hand, and to retain the saline solution. The sidewalls of the body may optionally contain a grip portion for the user to grip the container by hand, without the aid of a handle. Alternatively, the body may contain a handle for the user to grab onto while he or she dispenses the saline solution through the spout.

The neti pot can be transparent, opaque, or solid in appearance. The neti pot can be microwave safe to allow convenient heating of the contents within the neti pot. The container body may also include indicia.

The neti pot holds minimally 8 oz. of water, preferably at least 9 oz. of water, and at least more preferably 10 oz. of water. The neti pot optionally includes a marking on the exterior of the body to indicate the liquid level. The marking can be in any convenient form such as a printed line, a groove, a ring or the like.

The neti pot also includes a flattened platform circumferentially surrounding the opening and the threaded neck of the neti pot. The neck of the neti pot is fitted with a removable cap that has matching threads with the spiral thread on the neck. The diameter of the opening coincides with the diameter of an inner circular threaded portion of the cap. The cap closes the opening to provide an adequate liquid seal.

The spout is shaped to fit comfortably into nostrils of varying sizes. The spout can be formed from the same material as the neti pot, or of a different material. The neti pot optionally includes a plug or stopper fitted onto the spout, so that it retains the saline solution in the neti pot during the transportation of the neti pot. The connection of the plug to the spout can be of any convenient means including a compression-fit or threaded connection.

A cap is correspondingly configured to close the opening of the neti pot. The connection between the opening and the cap is a threaded engagement, in which the exterior of the cap includes a threaded portion which mates with the threaded neck. The cap can be made of materials selected from low-density polypropylene, high-density polypropylene, high-density polyethylene, wood, metal, ceramic, or any material that imparts sturdiness for the neti pot will be held and shaken by hand, and it should retain the saline solution with minimal or no leakage. While a threaded connection between the cap and body is preferred, other connections such as a compression type fitting can be used.

The cap has an outer rim and an inner circular rim that includes a threaded interior portion perpendicular to the top of the cap. A plurality of venting protrusions extend downward from the edge of either or both the outer and inner rims. Upon a full closure of the cap onto the body opening, tips of the venting protrusions contact the flattened surface of the platform of the neti pot. The contact of the venting protrusions with the platform provides a space between the bottom edges of the outer and inner rims and the surface of the platform. The spacing allows air to enter into the container body, but is small enough to provide sufficient seal between the cap and body to prevent spillage.

The cap optionally includes a plurality of gripping protrusions or indentions on its exterior for handling purposes. The number of gripping protrusions or indentions is optimized to achieve an adequate grip by hand. The number of exterior protrusions or indentions is about 4 to 8, preferably 6. The cap may also contain indicia.

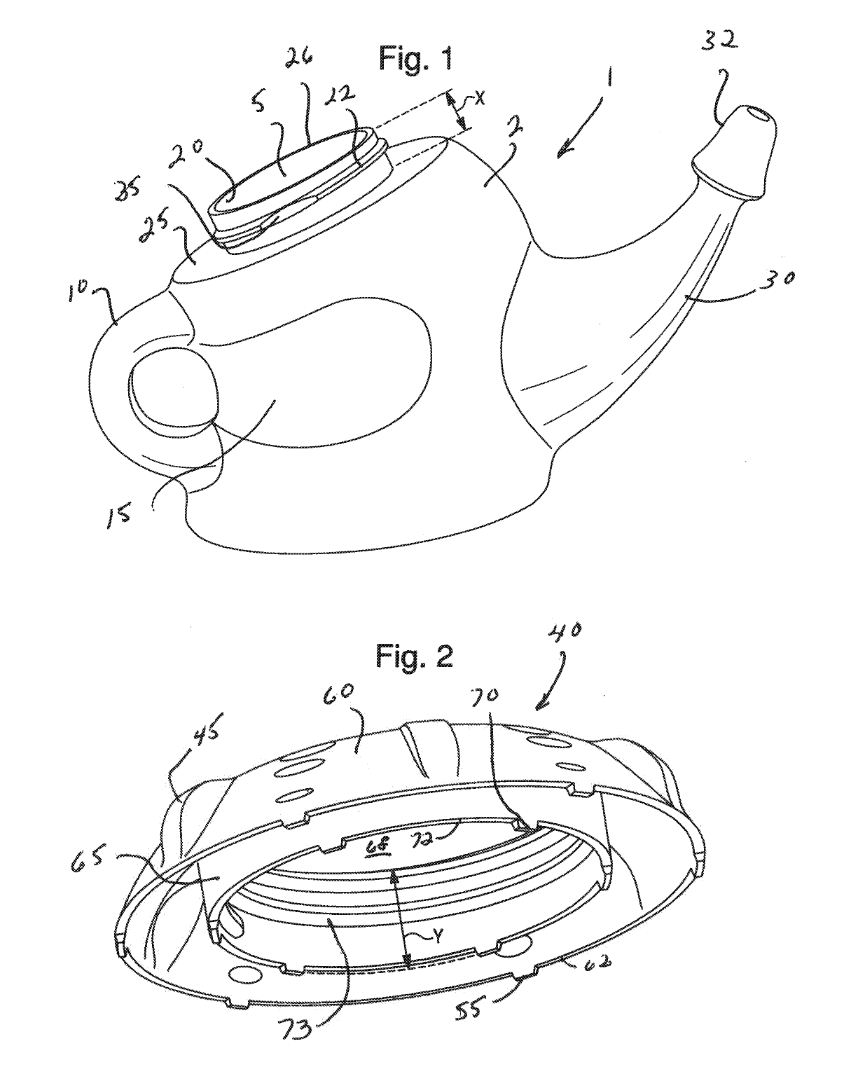

The neti pot can now be disclosed with respect to the Figures. FIG. 1 depicts a neti pot 1 comprising a body 2, an opening 5, a handle 10, a grip portion 15, and an extended pouring spout 30. The opening 5 is formed by a threaded neck 20. A flattened platform 25 is located on body 2 circumferentially around threaded neck 20. Threaded neck 20 has an exterior thread 22 circumventing the neck. Neck 20 has a height X, as shown in FIG. 1 from the platform 25 to the top 26 of neck 20. Thread 22 optionally includes at least one indention 35. The indentation 35 reduces the seal between thread 22 and matching threads from a removable cap, shown in FIG. 2. Spout 30 contains an end portion 32, which is shaped to fit within the nostrils.

FIG. 2 depicts a neti pot cap 40 comprising exterior gripping protrusions 45, an outer rim 60, and an inner circular rim 65, spaced from outer rim 60. Inner circular rim 65 has an interior thread 73 and is perpendicular to the top of cap 40.

Thread 73 matches thread 22 to secure cap 40 to body 2. Cap 40 further includes a plurality of venting protrusions 55 extending downward from the bottom edge 62 of outer rim 60, and/or a plurality of venting protrusions 70 extending downward from the bottom edge 72 of inner rim 65. Inner rim 65 has a height of Y, extending from the top 68 of cap 70 to the bottom edges of protrusions 55 and 70.

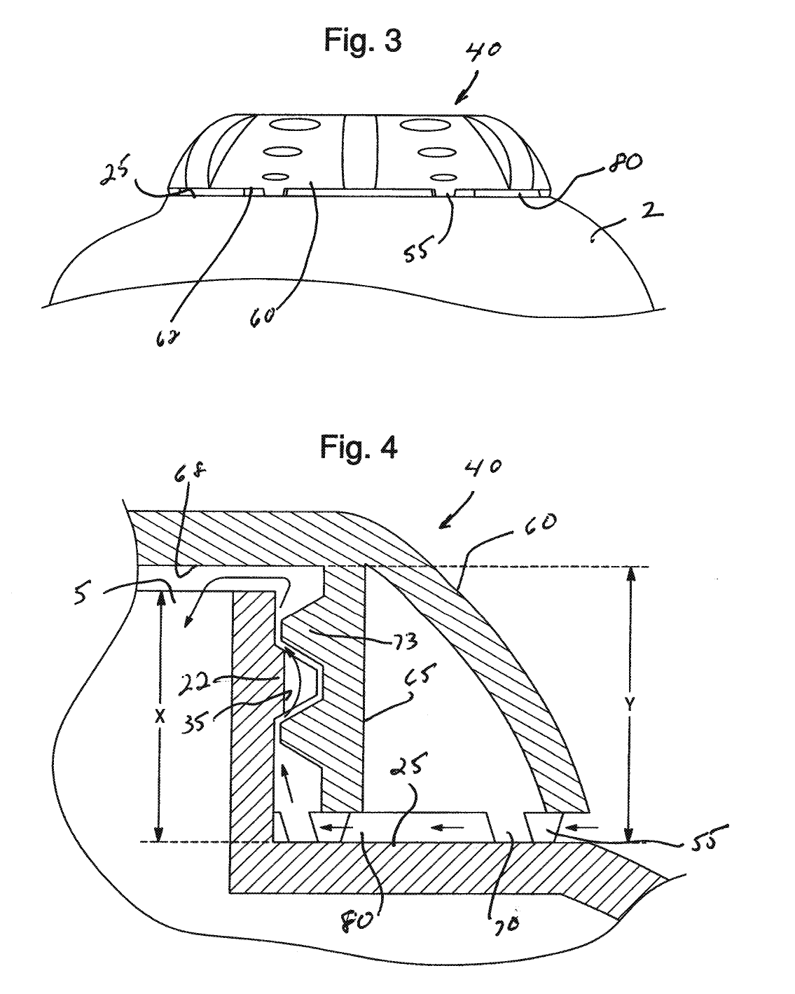

FIG. 3 depicts cap 40 atop neti pot 1 in a fully closed position. As can be seen, there is a minute space 80 between the platform 25 of body 2 and the bottom edges 62 and 72 (not shown) of outer rim 60 and inner rim 65 (not shown), respectively. The contact of protrusions 55 and 70 (not shown) with platform 25 provide an adequate seal between cap 40 and body 2 to prevent leakage of solution from neti pot 1. Space 80 allows air to enter the interior of neti pot 1 to allow ease of pouring from the pot.

FIG. 4 depicts a cross section view of FIG. 3 and illustrates the air flow into neti pot 1. As shown in FIG. 4, outer rim protrusions 55 and inner protrusions 70 elevate cap 40 from platform 25, to create an air channel 80. Air, shown by the arrows, enters the opening 5 of pot 1 through air channel 80. Optional indention 35 further augments air channel 80, such that air can travel between threads 73 and 22 and onwards to opening 5. The threaded portion of inner rim 65 is shown to have a height of Y, the threaded portion of neck 20 has a height of X, such that the height Y is greater than X to provide space 80 and allow air into opening 5.

### Method of Use

First remove cap 40 from neti pot 1, and fill neti pot 1 with 8 oz. of water. A dashed line (not shown) marked on the exterior of neti pot 1 indicates to a user when 8 oz. of water has been poured into neti pot 1.

A solid blend of a salt composition is emptied into neti pot 1. A particularly useful composition is one disclosed in co-pending, commonly assigned U.S. Application Attorney Docket No 99210, which includes a mixture of sodium chloride and sodium bicarbonate. It is to be understood a salt solution can be first formed and then poured into neti pot 1. Cap 40 is secured onto opening 5. The user plugs spout 30 by hand, a plug or a stopper (not shown). Neti pot 1 with cap 40 attached is then shaken by hand until the salt mixture is fully dissolved in the water.

Meanwhile, air enters air channel 80, and travels between threads 22 and 73, wherein the channel is optionally augmented by indention 35. Air then finally enters opening 5, ready to pressure the saline solution to exit through spout 30.

With neti pot 1 ready for use, the user bends forward to a comfortable level, tilts his or her head slightly up, holds neti pot 1 pointing downward (with opening 5 closed by cap 40 and spout 30 unobstructed) by handle 10 or grip portion 15, and applies spout 30 directly into his or her nostril. With the user's mouth open, neti pot 1 is then moved to force the saline solution to enter the user's nostril and nasal passage. During this process, the user has a full grip of neti pot with one hand, while the saline solution is being dispensed in a speed that is comfortable to the user without further manipulation. The process is repeated by applying spout 30 snugly against the other nostril. The saline solution that was flown into the nasal passages will drain from the nasal passages or the mouth and should not be swallowed by the user. The user then gently blows the nose. Any unused portion of the saline solution is discarded and neti pot 1 and cap 40 are then cleaned. A nasal rinse can be performed as often as needed or as recommended by a qualified physician.

Neti pot 1 and cap 40 should be thoroughly cleaned after each nasal rinse usage. Neti pot 1 and cap 40 can be placed in a dishwasher to be cleaned, or the assembly can be lathered with light soap then thoroughly rinsed under running water, then air-dry in an adequately ventilated area. Additionally, a vinegar and water solution can also be used to clean neti pot 1 and cap 40.

1. A neti pot for rinsing nasal passages comprising: a container body, an opening, a pouring spout, and a removable cap, wherein said cap comprises a rim with a bottom edge, and a plurality of venting protrusions extending downward from said edge, such that when said cap is placed over said opening and closed, a space is created between said body and the bottom edge of said rim.

2. The neti pot of claim 1, wherein said cap comprises an outer rim and an inner circular rim, and protrusions extending downward from a bottom edge of said inner circular rim, said outer rim or both.

3. The neti pot of claim 1, wherein said container body optionally includes a handle.

4. The neti pot of claim 1, wherein said container body optionally includes a grip portion.

5. The neti pot of claim 1, wherein said container body includes a platform circumferentially surrounding said opening, and said venting protrusions contacting said platform when said cap is in a closed position over said opening.

6. The neti pot of claim 2, wherein said container body further includes a neck defining said opening, said neck comprising a spiral thread, said inner circular rim containing a spiral thread which matches the spiral thread of said neck to mate said neck with said inner circular rim.

7. The neti pot of claim 6, wherein said spiral thread contains at least one indention to disrupt matching between said spiral threads on said neck and said inner circular rim.

8. The neti pot of claim 2, wherein the number of said venting protrusions located on either or both said outer rim or said inner circular rim is about 2 to 10.

9. The neti pot of claim 8, wherein the number of said venting protrusions located on either or both said outer rim or said inner circular rim is about 4 to 8.

10. The neti pot of claim 1, wherein said cap further includes a plurality of exterior gripping protrusions or indentions.

11. The neti pot of claim 10, wherein the number of exterior protrusions or indentions is about 4 to 8.

12. The neti pot of claim 2, wherein said venting protrusions extend from the bottom edge of said inner circular rim.

13. The neti pot of claim 1, wherein said container body is made from high-density polyethylene.

14. The neti pot of claim 13, wherein said spout is made from high-density polyethylene.

15. The neti pot of claim 13, wherein said cap is made from high-density polyethylene.

16. The neti pot of claim 1, wherein said container body is made of an inflexible material.

17. The neti pot of claim 1, wherein said pouring spout and said cap are made of said inflexible material.

18. The neti pot of claim 1, wherein said container body is made of a flexible material.
